(19) United States
(12) Patent Application Publication
(10) Pub. No.: US 2024/0161877 A1
(43) Pub. Date: May 16, 2024

(54) METHOD AND APPARATUS FOR PHYSICALLY ENCRYPTING NUCLEIC ACID

(71) Applicant: Onai Inc., San Jose, CA (US)

(72) Inventors: Guha Jayachandran, Cupertino, CA (US); Patrick Grinaway, Pawling, NY (US); Shriphani Palakodety, San Jose, CA (US); Galana Gebisa, Mountain View, CA (US)

(73) Assignee: Onai Inc., San Jose, CA (US)

(21) Appl. No.: 17/986,655

(22) Filed: Nov. 14, 2022

(51) Int. Cl. G16B 50/40

(52) U.S. Cl. CPC G16B 50/40

(57) ABSTRACT

An apparatus for physically encrypting nucleic acid, wherein the apparatus include at least a computing device configured to receive a nucleic acid sample from a subject, input a secret defined by the subject, generate a nucleic acid codebook as a function of the secret, wherein the nucleic acid codebook include a code pattern and a plurality of nucleic acid keys correlates to a plurality of nucleic acid values, and at least a manufacturing device configured to encrypt a nucleic acid sample received from a subject as a function of the nucleic acid codebook, and output an encrypted nucleic acid sample.

### FIELD OF THE INVENTION

The present invention generally relates to the field of cryptography. In particular, the present invention is directed to a method and an apparatus for physically encrypting nucleic acid.

### BACKGROUND

In recent years, genetic sequencing has dropped in cost and increased in commonality. It is used for medical purposes, tracing ancestry, law enforcement, and more. Nucleic acid sequences have substantial privacy implications. They not only identify individuals and provide personal information about those individuals but also reveal information about related persons. Existing technology does not adequately preserve the privacy of this information.

### SUMMARY OF THE DISCLOSURE

In an aspect, an apparatus for physically encrypting nucleic acid includes at least a computing device configured to receive a nucleic acid sample from a subject, input a secret defined by the subject, generate a nucleic acid codebook as a function of the secret, wherein the nucleic acid codebook includes a code pattern and a plurality of nucleic acid keys correlates to a plurality of nucleic acid values, and at least a manufacturing device configured to encrypt a nucleic acid sample received from a subject as a function of the nucleic acid codebook, and output an encrypted nucleic acid sample.

In another aspect, a method of nucleic acid physical encryption includes receiving, using a computing device, a nucleic acid sample from a subject. The method includes inputting, using the computing device, a secret defined by the subject. The method includes generating, using the computing device, a nucleic acid codebook as a function of the secret, wherein the nucleic acid codebook includes a code pattern and a plurality of nucleic acid keys correlates to a plurality of nucleic acid values. The method includes encrypting, using a manufacturing device, the nucleic acid sample as a function of the nucleic acid codebook. The method includes outputting, using the manufacturing device, an encrypted nucleic acid sample.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of an apparatus for physically encrypting nucleic acid;

FIG. 2 is a block diagram illustrating an exemplary embodiment of a nucleic acid decryption method;

FIG. 3 is a schematic diagram of an exemplary embodiment of a manufacturing device;

FIG. 4 is a flow diagram of an exemplary embodiment of a method of physically encrypting nucleic acid;

FIG. 5 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatuses and methods for physically encrypting nucleic acid; an apparatus may include at least a computing device configured to receive a nucleic acid sample from a subject, input a secret defined by the subject, generate a nucleic acid codebook as a function of the secret, wherein the nucleic acid codebook include a code pattern and a plurality of nucleic acid keys correlates to a plurality of nucleic acid values, and at least a manufacturing device configured to encrypt a nucleic acid sample received from a subject as a function of the nucleic acid codebook, and output an encrypted nucleic acid sample.

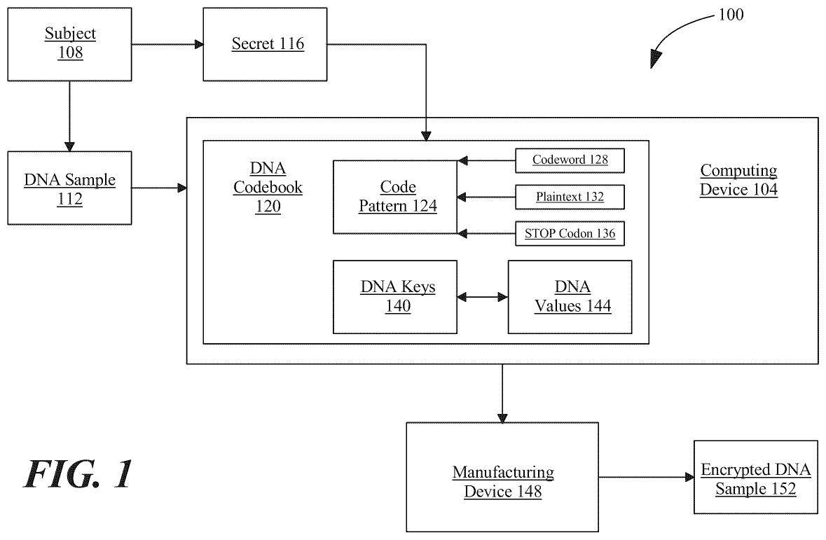

Referring now to FIG. 1, an exemplary embodiment of an apparatus for physically encrypting nucleic acid is illustrated. Apparatus may include a computing device 104. Computing device 104 may include any computing device as described in this disclosure, including without limitation a microcontroller, microprocessor, digital signal processor (DSP) and/or system on a chip (SoC) as described in this disclosure. Computing devices may include, be included in, and/or communicate with a mobile device such as a mobile telephone or smartphone. Computing device 104 may include a single computing device operating independently, or may include two or more computing devices operating in concert, in parallel, sequentially or the like; two or more computing devices may be included together in a single computing device or in two or more computing devices. Computing device 104 may interface or communicate with one or more additional devices as described below in further detail via a network interface devices. Network interface devices may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices, which may operate in parallel, in series, redundantly, or in any other manner used for distribution of tasks or memory between computing devices. Computing device 104 may be implemented using a “shared nothing” architecture in which data is cached at the worker. In an embodiment, this may enable scalability of apparatus 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, in an embodiment, methods and systems described herein may perform one or more operations on and/or involve a nucleic acid. As used in this disclosure, a “nucleic acid,” which may include deoxyribonucleic acid and/or one or more forms of ribonucleic acid, is a self-replicating material that is present in all living organisms to encode genes, including sequences of amino acids, “non-coding sequences” that can aid in transcription of nucleic acid into amino acid sequences, and the like. In some embodiments, nucleic acid may include two strands that coil around each other to form a double helix. Any strand of nucleic acid may be composed of simpler monomeric units such as nucleotides. Each nucleotide may include one of four nucleotides. In some cases, nucleotides may include, but are not limited to, cytosine (C), guanine (G), adenine (A), thymine (T), or the like thereof. In some cases, nucleotides may include a nitrogenous base. A plurality of nucleotides may join to one another in a chain through covalent bonds. Additionally, or alternatively, nitrogenous bases of two strands may be bound together as a function of a base pairing rule, wherein the base pairing rule may include A pair with T and C pair with G. Nucleic acid may include an RNA. As used in this disclosure, an “RNA,” also known as ribonucleic acid, is a polymeric molecule essential in a plurality of biological roles such as, without limitation, coding, decoding, regulation, expression of genes and the like thereof. In some embodiments, RNA may include a single strand folded onto itself. In some cases, RNA may include a messenger RNA, wherein the messenger RNA may be used to convey genetic information using nitrogenous bases such as guanine, uracil, adenine, and cytosine disrobed above in this disclosure. In some embodiments, RNA may be configured to, without any limitation, catalyzing biological reactions, controlling gene expression, sensing, and communicating responses to cellular signals or the like thereof. Additionally, or alternatively, RNA may direct a synthesis of proteins on a ribosome, wherein the synthesis may require use of transfer RNA (tRNA) molecules to deliver an amino acid to the ribosome. Amino acid may be linked together to form proteins as a function of ribosomal RNA (rRNA).

With continued reference to FIG. 1, computer device 104 is configured to receive a nucleotide sample 112, such as without limitation a nucleic acid sample, from a subject 108. As used in this disclosure, a “subject” is a living organism, which may include, without limitation, a human being, and/or a tissue sample therefrom. In some cases, subject 108 may be a human. In other cases, subject 108 may be an animal. As used in this disclosure, a “nucleic acid sample” is a sample containing nucleic acid which may be taken in the form of a fluid and/or tissue sample from subject, for instance and without limitation, swabbing tissue such as buccal tissue, nasal tissue, or the like, by extracting a sample of blood, lymph, semen, or other fluids of subject 108, by performing a biopsy, or the like. In some cases, nucleic acid sample 112 may include, without limitation, samples taken from drinking glasses, food with bite marks, bite marks on skin, and the like thereof. In an embodiment, nucleic acid sample 112 may include a nucleic acid sequence of subject 108. Nucleic acid sequences disclosed here will be described in further details below. In a non-limiting example, nucleic acid sample 112 may be a digital nucleic acid sample, wherein the digital nucleic acid sample is readable and/or mutable by computing device 104 using any processing step described in this disclosure, such as without limitation illumine dye sequencing or the like. Digital nucleic acid sample may include any genetic information and/or subject data within nucleic acid sample 112 described above in a data structure or data type such as, without limitation, vector, string, array, dictionary, tree, object, any data structure or data type described in this disclosure, and the like thereof. Digital nucleic acid sample may include any genetic information and/or subject data within nucleic acid sample 112 described above in an electronic format such as, without limitation, txt file, JSON file, XML, file, script, photographic file, any other electronic file, and the like thereof.

With continued reference to FIG. 1, computer device 104 is configured to input a secret 116. As used in this disclosure, a “secret” is an element of information not known and/or available to devices or persons generally; secret may, for instance, be known only to subject, to a small number of people intimate with subject, or the like. In an embodiment, computing device may generate secret automatically, for instance using a pseudorandom number generator, a random number generator, or the like; generation may include a key generation process, wherein the key generation process may include generating keys by random variation in selection of prime numbers, for instance for the purposes of a cryptographic system such as secret that relies prime factoring difficulty. Keys may be generated by randomized selection of parameters for a seed in a cryptographic system, such as elliptic curve cryptography, which is generated from a seed. Keys may be used to generate exponents for a cryptographic system such as Diffie-Helman or ElGamal that are based on the discrete logarithm problem. Secret may be used for an encryption process, for instance as described in further detail below. In some cases, secret 116 may be a session-specific secret. Session-specific secret may include a secret, which may be generated according to any process as described above, that uniquely identifies a particular instance of an attested boot and/or loading of a software monitor. Session-specific secret may include, without limitation, a random number. Session-specific secret may be converted to and/or added to a secure proof, verification datum, and/or key according to any process as described above for generation of a secure proof, verification datum, and/or key from a secret or “seed”; session-specific secret, a key produced therewith, verification datum produced therewith, and/or a secure proof produced therewith may be combined with module-specific secret, a key produced therewith, a verification datum produced therewith, and/or a secure proof produced therewith, such that, for instance, a software monitor and/or other signed element of attested boot and/or attested computing may include secure proof both of session-specific secret and of module-specific secret. In an embodiment, session-specific secret may be usable to identify that a given computation has been performed during a particular attested session, just as device-specific secret may be used to demonstrate that a particular computation has been produced by a particular device. This may be used, e.g., where computing device 104 or any component thereof is stateless, such as where any such element has no memory that may be overwritten and/or corrupted. In some cases, secret 116 may be defined by a subject 108. In other cases, secret 116 may be generated by computing device 104. In some embodiments, secret 116 may be kept unknown and/or unseen other than subject 108. In a non-limiting example, secret 116 may be a string defined by subject 108, wherein the string may include, without limitation, lowercase letters, uppercase letters, numbers, special characters, and the like thereof. Additionally, or alternatively, secret 116 may include a sequence length limitation, wherein subject 108 may be unable to define secret 116 exceeding a certain number of characters. In other embodiments, secret 116 may include no sequence length limitation. In a non-limiting example, a user may define a secret within a sequence length limitation, wherein the secret may remain unknown to others besides the user, and wherein the sequence length limitation may be 25 characters long. User may then input the secret to computing device 104. As used in this disclosure, an “encryption process” may be a process in a cryptographic system that alters, hides, or otherwise secures a fact, wherein the cryptographic system is a structure or scheme consisting of one or more algorithms that converts the fact into another form securely. In some cases, fact may be a “plaintext” which is intelligible when viewed in its intended format. In a non-limiting example, a cryptographic system may convert data from plaintext into a cyphertext, wherein the cyphertext is not intelligible when viewed in the same way as viewing plaintext. In some embodiments, encryption may involve use of a datum, known as an “encryption key,” to alter plaintext. Further, a cryptographic system may also convert cyphertext back into plaintext, which is a process known as “decryption.” In some embodiments, the decryption process may involve use of a datum, known as a “decryption key,” to return the cyphertext to its original plaintext form. In embodiments of cryptographic systems that are “symmetric,” decryption key may be essentially the same as encryption key: possession of either key makes it possible to deduce the other key quickly without further secret knowledge. In other embodiments, encryption and decryption keys in symmetric cryptographic systems may be kept secret and shared only with persons or entities that the user of the cryptographic system wishes to be able to decrypt the cyphertext. In a non-limiting example, a symmetric cryptographic system may be an Advanced Encryption Standard (“AES”), which arranges plaintext into matrices and then modifies the matrices through repeated permutations and arithmetic operations with an encryption key. Encryption and decryption disclosed here will be described in further details below.

With continued reference to FIG. 1, in embodiments of cryptographic systems that are “asymmetric,” either encryption or decryption keys cannot be readily deduced without additional secret knowledge, even given the possession of a corresponding decryption or encryption key, respectively; a common example is a “public key cryptographic system,” in which possession of the encryption key does not make it practically feasible to deduce the decryption key, so that the encryption key may safely be made available to the public. A non-limiting example of a public key cryptographic system is RSA, in which an encryption key involves the use of numbers that are products of very large prime numbers, but a decryption key involves the use of those very large prime numbers, such that deducing the decryption key from the encryption key requires the practically infeasible task of computing the prime factors of a number which is the product of two very large prime numbers. A further non-limiting example of an asymmetric cryptographic system may include a discrete-logarithm system based upon the relative ease of computing exponents mod a large integer, and the computational infeasibility of determining the discrete logarithm of resulting numbers absent previous knowledge of the exponentiations; an example of such a system may include Diffie-Hellman key exchange and/or public key encryption. Another non-limiting example is elliptic curve cryptography, which relies on the fact that given two points P and Q on an elliptic curve over a finite field, a definition of the inverse of a point −A as the point with negative y-coordinates, and a definition for addition where A+B=−R, the point where a line connecting point A and point B intersects the elliptic curve, where “0,” the identity, is a point at infinity in a projective plane containing the elliptic curve, finding a number k such that adding P to itself k times results in Q is computationally impractical, given correctly selected elliptic curve, finite field, and P and Q. A further example of asymmetrical cryptography may include lattice-based cryptography, which relies on the fact that various properties of sets of integer combination of basis vectors are hard to compute, such as finding the one combination of basis vectors that results in the smallest Euclidean distance. Embodiments of cryptography, whether symmetrical or asymmetrical, may include quantum-secure cryptography, defined for the purposes of this disclosure as cryptography that remains secure against adversaries possessing quantum computers; some forms of lattice-based cryptography, for instance, may be quantum-secure.

With continued reference to FIG. 1, computer device 104 is further configured to generate a nucleic acid codebook 120 as a function of secret 116. As used in this disclosure, a “nucleic acid codebook” is a codebook storing information regarding to nucleic acid sample 112, wherein the codebook is a data collection containing information intended to be complete and self-explanatory for each variable in a data file. In a non-limiting example, nucleic acid codebook 120 may be a document that stores cryptography codes for nucleic acid sample 112. In some embodiments, nucleic acid codebook may include methodological details of encrypting nucleic acid sample 112 such as, without limitation, steps, materials, instruments, parameters and the like thereof. In some embodiments, nucleic acid codebook 120 may include a one-time codebook. As used in this disclosure, a “one-time codebook” is a codebook with an expiration time. As used in this disclosure, an “expiration time” is a precise date and time at which nucleic acid codebook 120 and its derivatives are no longer valid. In some cases, without limitation, expiration time may be a date and time that nucleic acid codebook 120 is opened and closed. In some cases, without limitation, expiration time may be a date and time after nucleic acid codebook 120 is opened. In other cases, without limitation, expiration time may be a date and time after nucleic acid codebook 120 is generated by computing device 104. In a non-limiting example, a nucleic acid codebook 120 may be a one-time codebook, wherein the one-time codebook may include an expiration time 7 days after the nucleic acid codebook 120 is generated by computing device 104. Nucleic acid codebook 120 may not include any data after expiration time and/or may not be opened.

With continued reference to FIG. 1, nucleic acid codebook 120 includes a code pattern 124. As used in this disclosure, a “code pattern” is a guide for physically encrypting nucleic acid sample 112. In some embodiments, code pattern 124 may include a codeword 128. As used in this disclosure, a “codeword” is an element used for secrecy. In some cases, codeword may be a password defined by subject 108. In other cases, codeword may be secret 116. In other cases, codeword 128 may include letters, numbers, special characters and the like thereof. In some embodiments, code pattern 124 may include a plaintext 132. Plaintext may be any plaintext described in this disclosure. As used in this disclosure, a “plaintext” is a nucleic acid strand to be encrypted. In an embodiment, plaintext 132 may be a primer to hybridize with a nucleic acid strand to be encrypted. In a non-limiting example, plaintext 132 may include any nucleotides (i.e., nitrogenous bases) described above in this disclosure. In some embodiments, plaintext 132 may be included in an oligonucleotide, wherein the oligonucleotides may be produced through an application of polymerase. As used in this disclosure, an “oligonucleotide” is a short, single- or double-stranded nucleic acid or RNA molecule. In some cases, oligonucleotide may include, without limitation, antisense oligonucleotides (ASO), RNA interference (RNAi), aptamer RNA, and the like thereof. In some embodiments, oligonucleotides may be created as single stranded molecules with any user-specified sequence, such as plaintext 132. As used in this disclosure, a “Polymerase” is an enzyme that synthesizes long chains of polymers or nucleic acids. In some embodiments, polymerase may include nucleic acid polymerase to assemble nucleic acid molecules by copying a nucleic acid template strand as a function of base pairing rule. In other embodiments, polymerase may include RNA polymerase to assemble RNA molecules through a half ladder replication. Further, code pattern 124 may include a STOP codon 136 configured to terminate the encryption. As used in this disclosure, a “STOP codon” is a codon that signals a termination of a translation process of current protein. In some cases, codon may be a genetic code, wherein the genetic code may be used by living cells to translate information encoded within nucleic acid or RNA into proteins. As used in this disclosure, to “translate” means to synthesize proteins from ribosomes in cytoplasm or endoplasmic reticulum after transcription of nucleic acid to RNA in a cell's nucleus. In some cases, STOP codon 136 may be a nucleotide triplet within messenger RNA (mRNA). In some embodiments, STOP codon 136 may signal termination of translation by as a function of a release factor, wherein the release factor is a protein that allows for termination of translation by recognizing STOP codon 136 in mRNA and releasing new peptides from ribosomes. In a non-limiting example, STOP codon 136 may include UAG, UAA, UGA, other triplets, and the like thereof. In some embodiments, STOP codon 136 may not be recognized by tRNA as disclosed above. In a non-limiting example, code pattern 124 may be predefined as “STOP codon codewordIplaintext” in a sequence.

With continued reference to FIG. 1, nucleic acid codebook 120 includes a plurality of nucleic acid keys 140 correlates to a plurality of nucleic acid values 144. As used in this disclosure, a “nucleic acid key” is a key containing true information regarding to nucleic acid sample 112. In some embodiments, each nucleic acid key of plurality of nucleic acid keys 140 may include a nucleic acid sequence element of a nucleic acid sample 112, wherein the nucleic acid sequence element is one of four nucleotides of nucleic acid as disclosed above in this disclosure. In an embodiment, nucleic acid sequence element may be a base of nucleic acid. In other cases, each nucleic acid key of plurality of nucleic acid keys 140 may include elements from plaintext 132. As used in this disclosure, a “nucleic acid value is a value that contains in depth detail corresponding to a given nucleic acid key 140. In some embodiments, each nucleic acid value of plurality of nucleic acid values 144 may include an encrypted code, wherein the encrypted code is a code that hides true information within nucleic acid key 140. In some cases, each nucleic acid value of plurality of nucleic acid values 140 may include codeword 128. In some embodiments, without limitation, nucleic acid codebook may include a plurality of nucleic acid key value pairs, wherein each nucleic acid key value pair may include nucleic acid key 140 mapped to nucleic acid value 144. Additionally, or alternatively, nucleic acid codebook 120 may include a term index. As used in this disclosure, a “term index” is a data structure to facilitate fast lookup of elements in a collection of data (i.e., index). In some cases, term index may use a zero-based indexing, wherein the zero-based indexing may configure plurality of nucleic acid keys 140 of nucleic acid codebook 120 to start with index 0. In some cases, term index may use a one-based indexing, wherein the one-based indexing may configure plurality of nucleic acid keys 140 of nucleic acid codebook 120 to start with index 1. In other cases, term index may use an n-based indexing, wherein the n-based indexing may configure plurality of nucleic acid keys 140 of nucleic acid codebook 120 to start with any index.

With continued reference to FIG. 1, in some embodiments, nucleic acid codebook 120 may include one or more cryptographic hashes. As used in this disclosure, a “cryptographic hash” is a mathematical representation data within nucleic acid codebook 120; the mathematical representation is produced by a lossy “one-way” algorithm known as a “hashing algorithm.” Hashing algorithm may be a repeatable process; that is, identical lots of data may produce identical hashes each time they are subjected to a particular hashing algorithm. Because hashing algorithm is a one-way function, it may be impossible to reconstruct a lot of data from a hash produced from the lot of data using the hashing algorithm. In the case of some hashing algorithms, reconstructing the full lot of data from the corresponding hash using a partial set of data from the full lot of data may be possible only by repeatedly guessing at the remaining data and repeating the hashing algorithm; it is thus computationally difficult if not infeasible for a single computer to produce the lot of data, as the statistical likelihood of correctly guessing the missing data may be extremely low. However, the statistical likelihood of a computer of a set of computers simultaneously attempting to guess the missing data within a useful timeframe may be higher, permitting mining protocols as described in further detail below.

With continued reference to FIG. 1, in an embodiment, hashing algorithm may demonstrate an “avalanche effect,” whereby even extremely small changes to lot of data produce drastically different hashes. This may thwart attempts to avoid the computational work necessary to recreate a hash by simply inserting a fraudulent datum in data lot, enabling the use of hashing algorithms for “tamper-proofing” data such as data contained in an immutable ledger as described in further detail below. This avalanche or “cascade” effect may be evinced by various hashing processes; persons skilled in the art, upon reading the entirety of this disclosure, will be aware of various suitable hashing algorithms for purposes described herein. Verification of a hash corresponding to a lot of data may be performed by running lot of data through a hashing algorithm used to produce the hash. Such verification may be computationally expensive, albeit feasible, potentially adding up to significant processing delays where repeated hashing, or hashing of large quantities of data, is required, for instance as described in further detail below. Examples of hashing programs include, without limitation, SHA256, a NIST standard; further current and past hashing algorithms include Winternitz hashing algorithms, various generations of Secure Hash Algorithm (including “SHA-1,” “SHA-2,” and “SHA-3”), “Message Digest” family hashes such as “MD4,” “MD5,” “MD6,” and “RIPEMD,” Keccak, “BLAKE” hashes and progeny (e.g., “BLAKE2,” “BLAKE-256,” “BLAKE-312,” and the like), Message Authentication Code (“MAC”)-family hash functions such as PMAC, OMAC, VMAC, HMAC, and UMAC, Poly1305-AES, Elliptic Curve Only Hash (“ECOH”) and similar hash functions, Fast-Syndrome-based (FSB) hash functions, GOST hash functions, the Grøstl hash function, the HAS-160 hash function, the JH hash function, the RadioGatún hash function, the Skein hash function, the Streebog hash function, the SWIFFT hash function, the Tiger hash function, the Whirlpool hash function, or any hash function that satisfies, at the time of implementation, the requirements that a cryptographic hash be deterministic, infeasible to reverse-hash, infeasible to find collisions, and have the property that small changes to an original message to be hashed will change the resulting hash so extensively that the original hash and the new hash appear uncorrelated to each other. A degree of security of a hash function in practice may depend both on the hash function itself and on characteristics of the message and/or digest used in the hash function. For example, where a message is random, for a hash function that fulfills collision-resistance requirements, a brute-force or “birthday attack” to detect collision may be on the order of O(2^{2n/2}) for n output bits; thus, it may take on the order of 2²⁵⁶ operations to locate a collision in a 312 bit output. “Dictionary” attacks on hashes likely to have been generated from a non-random original text can have a lower computational complexity, because the space of entries they are guessing is far smaller than the space containing all random permutations of bits. However, the space of possible messages may be augmented by increasing the length or potential length of a possible message, or by implementing a protocol whereby one or more randomly selected strings or sets of data are added to the message, rendering a dictionary attack significantly less effective.

With continued reference to FIG. 1, apparatus 100 may further include at least a manufacturing device 148. Manufacturing device 148 may be an automated manufacturing system, wherein the automated manufacturing system may include computing device 104 and any components that are used for performing processing steps including CRISPR, illumina dye sequencing technique as described in this disclosure. Manufacturing device 148 may include an additive manufacturing device, and/or any device designed or configured to produce a component, product, or the like using an additive manufacturing process, in which material is deposited on the workpiece to be turned into the finished result. In some embodiments, an additive manufacturing process is a process in which material is added incrementally to a body of material in a series of two or more successive steps. The material may be added in the form of a stack of incremental layers; each layer may represent a cross-section of the object to be formed upon completion of the additive manufacturing process. Each cross-section may, as a non-limiting example, be modeled on a computing device as a cross-section of graphical representation of the object to be formed; for instance, a computer aided design (CAD) tool may be used to receive or generate a three-dimensional model of the object to be formed, and a computerized process may derive from that model a series of cross-sectional layers that, when deposited during the additive manufacturing process, together will form the object. The steps performed by an additive manufacturing system to deposit each layer may be guided by a computer aided manufacturing (CAM) tool. In other embodiments, a series of layers are deposited in a substantially radial form, for instance by adding a succession of coatings to the workpiece. Similarly, the material may be added in volumetric increments other than layers, such as by depositing physical voxels in rectilinear or other forms. Additive manufacturing, as used in this disclosure, may specifically include manufacturing done at the atomic and nano level. Additive manufacturing also includes bodies of material that are a hybrid of other types of manufacturing processes, e.g. forging and additive manufacturing as described above. As an example, a forged body of material may have welded material deposited upon it which then comprises an additive manufactured body of material.

With continued reference to FIG. 1, in some embodiments, manufacturing device 148 may include a subtractive manufacturing device, which may perform one or more subtractive manufacturing processes. One or more steps may include a subtractive manufacturing process, which produces the product by removing material from a workpiece; the removal of material may be accomplished using abrasives, cutting tools or endmills, laser cutting or ablation, removal using heat, or any other method that removes material from the workpiece. Each subtractive manufacturing process used may be any suitable process, such as, but not limited to, rotary-tool milling, electrical discharge machining, ablation, etching, erosion, cutting, sawing, sanding, polishing, grinding, and cleaving, among others. If rotary-tool milling is utilized, this milling may be accomplished using any suitable type of milling equipment, such as milling equipment having either a vertically or horizontally oriented spindle shaft. Examples of milling equipment include bed mills, turret mills, C-frame mills, floor mills, gantry mills, knee mills, and ram-type mills, among others. In some embodiments, the milling equipment used for removing material may be of the computerized numerical control (CNC) type that is automated and operates by precisely programmed commands that control movement of one or more parts of the equipment to affect the material removal. CNC machines, their operation, programming, and relation to CAM tools and CAD tools are well known and need not be described in detail herein for those skilled in the art to understand the scope of the present invention and how to practice it in any of its widely varying forms. Further, subtractive manufacturing may be performed using spark-erosive devices; for instance, subtractive manufacturing may include removal of material using electrical discharge machining (EDM). EDM may include wire EDM, plunge EDM, immersive EDM, ram EDM, or any other EDM manufacturing technique. Subtractive manufacturing may be performed using laser-cutting processes. Subtractive manufacturing may be performed using water-jet or other fluid-jet cutting techniques. Fundamentally, any process for removal of material may be employed for subtractive manufacturing.

With continued reference to FIG. 1, in some embodiments, manufacturing device 148 may include a chemical instrument. As used in this disclosure, a “chemical instrument” is a device or tool used for scientific purposes, particularly chemical purposes. In a non-limiting example, chemical instrument may be configured to perform chemical reactions. In some cases, manufacturing device 148 may include a plurality of chemical instruments. Plurality of chemical instruments may vary in size, shape, purpose, complication, complexity, and the like thereof. In a non-limiting example, chemical instrument may be a simple laboratory equipment such as, without limitation, scales, rulers, chronometers, thermometers, and the like thereof. In another non-limiting example, chemical instrument may be a chemical analysis equipment such as, without limitation, benchtop pH meter, discrete analyzer, elemental analyzer, reactor, and the like thereof. In some embodiments, chemical instrument may include integration of computing device such as computing device 104 to improve and simplify operational control. In some cases, integration of computing device 104 may enhance and extend instrumental functions, conditions, parameter adjustments, data processing, and the like of chemical instrument and/or manufacturing device 148. In a non-limiting example, a manufacturing device 148 may communicatively connected to computing device 104, wherein the manufacturing device 148 may include a plurality of chemical instruments configured to perform chemical reaction such as, without limitation, DNA oligonucleotide synthesis. In this case, manufacturing device 148 may be a DNA synthesizer.

With continued reference to FIG. 1, manufacturing device 148 is configured to encrypt nucleic acid sample 112 as a function of nucleic acid codebook 120, wherein encrypting further include replacing at least a sequence of nucleic acid sample 112 with a code pattern by matching a key. As used in this disclosure, “encrypting nucleic acid sample” means hiding or perplexing genetic information by a computational method in order to improve genetic privacy in nucleic acid sequencing process. Encryption process may be any encryption process described in current disclosure. In a non-limiting example, computing device 104 may receive a nucleic acid sample 112, wherein the nucleic acid sample 112 may include a main strand and a complementary strand, wherein both the main strand and the complementary strand include a plurality of nucleotides of nucleic acid sample. Plurality of nucleotides of main strand may correlate with plurality of nucleotides of complementary strand according to base pairing rule described above in this disclosure. Nucleic acid sample may further be computationally expressed as a first array “[T, G, A, C]” and a second array “[A, C, T, G],” wherein the first array may represent main strand of nucleic acid sample and the second array may represent complementary strand of nucleic acid sample. A user may input a secret “T:w; A:x; G:y; C:z,” wherein the secret may include a plurality of user defined passwords (i.e., codeword 128) such as w, x, y, and z, in any length match with a plurality of sequences (i.e., plaintext 132) of nucleic acid sample 120. Computing device 104 may then generate a nucleic acid codebook 120 as a function of secret, wherein the nucleic acid codebook 120 may include a code pattern 124 and a nucleic acid dictionary. Code pattern 124 may be expressed as a sequential combination of codeword 128, plaintext 132, and stop codon 136: “wxyz|TAGC|AC.” Nucleic acid dictionary within nucleic acid codebook 120 may be generated by computing device 104 as a function of base pairing rule: “{{T, A}, {G, C}, {A, T}, {C, G}}”. Nucleic acid Dictionary may be any dictionary described in this disclosure. Nucleic acid dictionary may include a plurality of key value pairs, wherein each key value pair (i.e., {T, A}, {G, C}, {A, T}, and {C, G}) may include a single nucleotide from main strand/first array as key correlated to a single nucleotide from complementary strand/-second array as value. Computing device 104 may further encrypt nucleic acid sample 112 as a function of nucleic acid codebook 120 by replacing one or more sequences within nucleic acid sample 112 with codeword 128 within code pattern 124 by matching keys and/or values in nucleic acid dictionary with code pattern 124 util reaching stop codon 136. Encrypted nucleic acid sample 152 after encryption may be expressed as “[[w, y], [x, z]].”

With continued reference to FIG. 1, in some embodiments, encrypting nucleic acid sample 112 may include encrypting nucleic acid sample 112 as a function of a Clustered Regularly Interspaced Short Palindromic Repeats (CRISPR) technology. As used in this disclosure, a “CRISPR” is programmable technology that targets specific stretches of genetic code to edit nucleic acid at precise locations. CRISPR technology may include CRISPR-CAS 9. Cas9 (or “CRISPR-associated protein 9”) is an enzyme that uses CRISPR sequences as a guide to recognize and cleave specific strands of nucleic acid that are complementary to the CRISPR sequence. Cas9 enzymes together with CRISPR sequences form the basis of a technology known as CRISPR-Cas9 that can be used to edit genes within organisms. CRISPR technology may include Class 1 CRISPR systems including type I (cas3), type III (cas10), and type IV and 12 subtypes. CRISPR technology may include Class 2 CRISPR systems including type II (cas9), type V (cas12), type VI (cas13), and 9 subtypes. In some embodiments, CRISPR technology may involve CRISPR-Cas design tools which are computer software platform 100s and bioinformatics tools used to facilitate the design of guide RNAs (gRNAs) for use with the CRISPR/Cas gene editing system. For example, CRISPR-Cas design tools may include: CRISPRon, CRISPRoff, Invitrogen TrueDesign Genome Editor, Breaking-Cas, Cas-OFFinder, CASTING, CRISPy, CCTop, CHOPCHOP, CRISPOR, sgRNA Designer, Synthego Design Tool, and the like. CRISPR technology may also be used as a diagnostic tool. For example, CRISPR-based diagnostics may be coupled to enzymatic processes, such as SHERLOCK-based Profiling of IN vitro Transcription (SPRINT). SPRINT can be used to detect a variety of substances, such as metabolites in patient samples or contaminants in environmental samples, with high throughput or with portable point-of-care devices. In a non-limiting example, encrypting nucleic acid sample 112 may include designing a gRNA based on subject-defined location of nucleic acid sample 112 using computing device 104, and cutting nucleic acid sample 112 as a function of the gRNA using Cas9, wherein the gRNA is a guide RNA is a short synthetic RNA that functions as a guide for nucleic-acid-targeting enzymes. After cutting nucleic acid sample 112, only a piece of nucleic acid sample 112 with a sequence that matches gRNA remains in nucleic acid sample 112.

With continued reference to FIG. 1, in some embodiments, encrypting nucleic acid sample 112 may include encrypting nucleic acid sample 112 as a function of an illumina dye sequencing technique. As used in this disclosure, an “illumina dye sequencing technique” is a technique used to determine a series of base pairs in nucleic acid (i.e., nucleic acid sequencing). In some embodiments, illumina dye sequencing technique may include fragmenting nucleic acid, wherein fragmenting nucleic acid may include adding a plurality of adapters containing a plurality of segments that act as a plurality of reference points throughout entire process of illumina dye sequencing technique. In some embodiments, fragmented nucleic acid may be loaded onto a flow cell, wherein the flow cell contains nano cells that space out fragmented nucleic acid. In some cases, flow cell may also help with overcrowding of fragmented nucleic acid. Additionally, or alternatively, flow cell may contain oligonucleotides that provide an anchoring point for plurality of adapters to attach. In some embodiments, illumina dye sequencing technique may include generating a cluster after fragmented nucleic acid attached to flow cell. In some cases, generating a cluster may include copying fragmented nucleic acid a plurality of times as a function of a bridge amplification PCR. In some embodiments, illumina dye sequencing technique may include a chip, wherein the chip acts as a placeholder for a plurality of primers and a plurality of modified nucleotides. In some cases, a plurality of modified nucleotides may include a reversible fluorescent blocker, wherein the reversible fluorescent blocker only allows nucleic acid polymerase to add one modified nucleotide at a time onto fragmented nucleic acid. Further, illumina dye sequencing technique may include output of a photo of a chip. In some embodiments, computing device 104 may determine base has been added during illumina dye sequencing as a function of a wavelength of a fluorescent tag, wherein the fluorescent tag is a fluorophore that used to determine distribution and quantity of a particular target biomolecule both in vitro and in vivo. In other embodiments, computing device 104 may record chip. In a non-limiting example, encrypting nucleic acid sample 112 may include selecting a piece of nucleic acid sample 112 by subject 108. Computing device 104 may then be configured to cut nucleic acid sample 112 as a function of the subject-selected piece using CRISPR as described above in this disclosure. Clipped nucleic acid sample may be further encrypted as a function of nucleic acid codebook 120, wherein encrypting clipped nucleic acid sample may further include replacing sequence of clipped nucleic acid sample with code pattern within nucleic acid codebook by matching nucleic acid key as described above in this disclosure. Computing device 104 may then initiate an illumina dye sequencing process to synthesize encrypted nucleic acid sample on a chip, such as, without limitation, microfluidics chip, photonics chip, and the like thereof.

With continued reference to FIG. 1, manufacturing device 148 is further configured to output an encrypted nucleic acid sample 152. As used in this disclosure, an “encrypted nucleic acid sample” is a nucleic acid sample with hidden genetic information. In some embodiments, encrypted nucleic acid sample 152 may be unable to obtain true information about nucleic acid sample 112 such as genetic information regarding to subject 108 without secret 116 defined by subject 108 prior to the encryption. In some embodiments, encrypted nucleic acid sample 152 may be a physical picture, card, chip, or any other physical object and the like thereof. In a non-limiting example, outputting encrypted nucleic acid sample 152 may include outputting microfluidic chip and/or photonic chip containing encrypted nucleic acid sample which produced through illumina dye sequencing as described above. In some embodiments, outputting encrypted nucleic acid sample 152 may include a deposition of material in additive manufacturing processes, wherein the deposition of material may be accomplished by any suitable means. Deposition may be accomplished using stereolithography, in which successive layers of polymer material are deposited and then caused to bind with previous layers using a curing process such as curing using ultraviolet light. Additive manufacturing processes may include “three-dimensional printing” processes that deposit successive layers of power and binder; the powder may include polymer or ceramic powder, and the binder may cause the powder to adhere, fuse, or otherwise join into a layer of material making up the body of material or product. Additive manufacturing may include metal three-dimensional printing techniques such as laser sintering including direct metal laser sintering (DMLS) or laser powder-bed fusion. Likewise, additive manufacturing may be accomplished by immersion in a solution that deposits layers of material on the body of material, by depositing and sintering materials having melting points such as metals, such as selective laser sintering, by applying fluid or paste-like materials in strips or sheets and then curing that material either by cooling, ultraviolet curing, and the like, any combination of the above methods, or any additional methods that involve depositing successive layers or other increments of material. Methods of additive manufacturing may include without limitation vat polymerization, material jetting, binder jetting, material extrusion, fused deposition modeling, powder bed fusion, sheet lamination, and directed energy deposition. Methods of additive manufacturing may include adding material in increments of individual atoms, molecules, or other particles. An additive manufacturing process may use a single method of additive manufacturing or combine two or more methods.

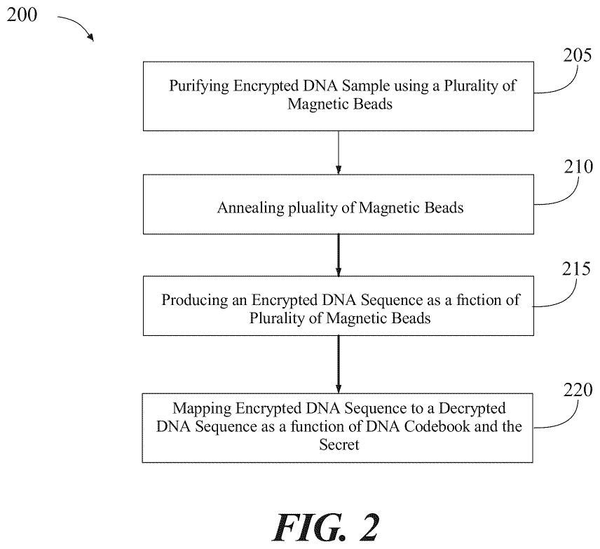

With continued reference to FIG. 1, in some embodiments, outputting encrypted nucleic acid sample 152 may include decrypting encrypted nucleic acid sample 152 as a function of a nucleic acid decryption method. As used in this disclosure, a “nucleic acid decryption method” is a method for decrypting encrypted nucleic acid sample 152. Decryption may include any decryption described in this disclosure. In some embodiments, nucleic acid decryption method may include a plurality of steps for obtaining hidden true information such as genetic information of Subject 108 within encrypted nucleic acid sample 152. Nucleic acid decryption method disclosed here will be described in further details below in reference to FIG. 2. In other embodiments, decrypting encrypted nucleic acid sample 152 may include decrypting encrypted nucleic acid sample 152 as a function of a photo-cleavable site. As used in this disclosure, a “photocleavable site,” also known as photolabile site, photoremovable site, photosensitive site, and the like, is a site with high chemoselectivity that allow researchers to control spatial, temporal and concentration variables with light. In some cases, photocleavable site may include a traceless reagent process. In a non-limiting example, photocleavable site may be used in multistep organic syntheses such as generating a decrypted nucleic acid sample as a function of the encrypted nucleic acid sample 152 and codebook 120 along with secret 116 defined by subject 108. Additionally, or alternatively, decrypting encrypted nucleic acid sample 152 may include decrypting encrypted nucleic acid sample 152 as a function of CRISPR which is described above in this disclosure.

Now referring to FIG. 2, an exemplary embodiment of a nucleic acid decryption method 200 is illustrated. In some embodiments, nucleic acid decryption method 200 may include a step 205 of purifying encrypted nucleic acid sample 152 using a plurality of magnetic beads. As used in this disclosure, a “magnetic bead” is a bead made up of 2030 nm iron oxide particles with superparamagnetic properties. In some cases, each magnetic bead of plurality of magnetic beads may be composed by a plurality of magnetite particles (Fe₃O₄). In some embodiments, plurality of magnetic beads may display magnetic behavior only in presence of an external magnetic field. In other embodiments, plurality of magnetic beads may not exhibit magnetic behavior without presence of external magnetic field. In some cases, plurality of magnetic beads may be used for isolating and/or purifying nucleic acid, RNA, proteins, other biomolecules and the like thereof. In a non-limiting example, after plurality of magnetic beads bind encrypted nucleic acid sample 152, an external magnetic field makes plurality of magnetic beads stick to an outer edge of a containing tube. As plurality of magnetic beads are immobilized, bead-bound nucleic acid may be retained during washing. In another non-limiting example, encrypted nucleic acid sample 152 may be in a solution and encrypted may be selectively bound to the surface of plurality of magnetic beads while other contaminants stay in the solution. In some cases, solution may be a nucleic acid mixture. In some embodiments, step 205 of purifying encrypted nucleic acid sample 152 may require a lesser number of steps and reagents than other nucleic acid extraction processes.

With continued reference to FIG. 2, nucleic acid decryption method 200 may include a step 210 of annealing plurality of magnetic beads. As used in this disclosure, “annealing” means boiling plurality of magnetic beads and allowing plurality of magnetic beads to reset to a lower temperature or original temperature. In some cases, annealing plurality of magnetic beads may result in better encrypted nucleic acid sequence than washing. Encrypted nucleic acid sequence disclosed here will be described in further detail below. In some embodiments, step 210 of annealing plurality of magnetic beads may include heating a plurality of magnetic beads at a first temperature and cooling the plurality of magnetic beads at a second temperature, wherein the second temperature is different than the first temperature. In some cases, first temperature may be lower than 100 Celsius. In other cases, second temperature may be lower than first temperature. In some embodiments, heating a plurality of magnetic beads may include applying heat to bead-bound nucleic acid until encrypted nucleic acid sample 152 on plurality of magnetic beads is separated into two single strands. In other embodiments, heating plurality of magnetic beads may include a nucleic acid denaturation. As used in this disclosure, a “nucleic acid denaturation” is a process when a nucleic acid mixture is heated enough, double-stranded nucleic acid unwinds, and hydrogen bonds that hold two strands together weaken and finally break. In some embodiments, cooling plurality of magnetic beads may allow plurality of nucleic acid molecules to be formed in nucleic acid mixture. In some cases, plurality of nucleic acid molecules may be used to produce certain nucleic acid sequences.

With continued reference to FIG. 2, nucleic acid decryption method 200 may include a step 215 of producing an encrypted nucleic acid sequence as a function of plurality of magnetic beads. As used in this disclosure, an “encrypted nucleic acid sequence” is a nucleic acid sequence with encrypted genetic information regarding to subject 108. Nucleic acid sequence may be any nucleic acid sequence disclosed in this disclosure. In a non-limiting example, encrypted nucleic acid sequence of encrypted nucleic acid sample 152 may be separated from surface of plurality of magnetic beads after step 210 of annealing plurality of magnetic beads. In some embodiments, step 215 of producing encrypted nucleic acid sequence may include cutting a nucleic acid overhang from encrypted nucleic acid sample 152. As used in this disclosure, a “nucleic acid overhang” is a stretch of unpaired nucleotides (i.e., single strand) in the end of encrypted nucleic acid sample 152. In some cases, nucleic acid overhang may include a cohesive end or sticky end, wherein the cohesive end or sticky end may be created by restriction endonucleases when restriction endonucleases cut encrypted nucleic acid sample 152.

With continued reference to FIG. 2, nucleic acid decryption method 200 may include a step 220 of mapping encrypted nucleic acid sequence to a decrypted nucleic acid sequence as a function of nucleic acid codebook 120 along with secret 116 defined by subject 108. As used in this disclosure, a “decrypted nucleic acid sequence” is a nucleic acid sequence with true genetic information regarding to subject 108. Nucleic acid sequence may be any nucleic acid sequence described in this disclosure. In some cases, decrypted nucleic acid sequence may be plaintext 132 within codebook 120. In some embodiments, decrypted nucleic acid sequence may be identical to nucleic acid sequence of nucleic acid sample 112 received from subject 108 as described above in reference to FIG. 1. In other embodiments, mapping encrypted nucleic acid sequence to a decrypted nucleic acid sequence may include validating secret 116 against codeword 128 within codebook 120. Further, codebook 120 may be no longer valid after step 220 of mapping encrypted nucleic acid sequence to decrypted nucleic acid sequence. In some embodiments, manufacturing device 148 may be configured to output a physical decrypted nucleic acid sequence similar to encrypted nucleic acid sample as disclosed above in reference to FIG. 1.

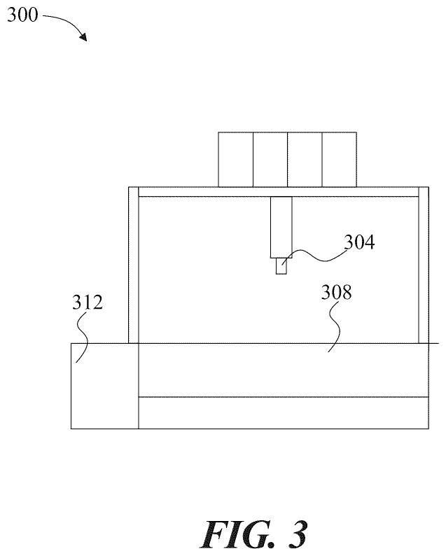

Referring now to FIG. 3, a block diagram of an exemplary embodiment of an apparatus 100 is illustrated. Apparatus 100 may include at least an applicator 304. At least an applicator 304 may include any device used to deposit layers of ingredient or material. For instance, applicator 304 may include a printer head for a 3D printer. Applicator 304 may include an extruding device for extruding fluid or paste material, a sprayer or other applicator for bonding material, an applicator for powering, a sintering device such as a laser, or other such material. Applicator 304 may draw upon one or more reservoirs of liquid, paste, and/or powdered materials, which may advance such materials to application using, without limitation, auger screws, pistons, gravity, and/or pressure.

Continuing to view FIG. 3, apparatus 100 may include a workpiece support 308. Workpiece support 308 may be a structure that supports a workpiece during the one or more manufacturing steps. Workpiece support 308 may include a base table. Base table may include a surface to which a workpiece or other components may be secured. Surface may be oriented horizontally, vertically, or in any other orientation. Surface may be substantially planar. Workpiece support 308 may include a substrate for initial deposition of material in an additive process.

Still referring to FIG. 3, apparatus 100 may include a powered manufacturing device 300. As used herein, a powered manufacturing device 300 is a manufacturing device 300 in which at least one component of the manufacturing device 300 includes at least a component powered by something other than human power. At least a component may be powered by any non-human source, including without limitation electric power generated or stored by any means, heat engines including steam, internal combustion, or diesel engines, wind power, waterpower, pneumatic power, or hydraulic power. Powered components may include any components of manufacturing device 300. Applicator 304 may be powered; for instance, applicator 304 may include an endmill mounted on a spindle rotated by a motor (not shown). Workpiece support 308 may be powered. Where manufacturing device 300 is a mechanical device, motion of components along linear or rotary constraints may be powered; for instance, motion of base table along one or more linear constraints such as linear slides may be driven by a motor or other source of power. Similarly, rotation of rotary table may be driven by a power source. Tool-changer, where present, may be driven by power. In some embodiments, all or substantially all of the components of manufacturing device 300 are powered by something other than human power; for instance, all components may be powered by electrical power.

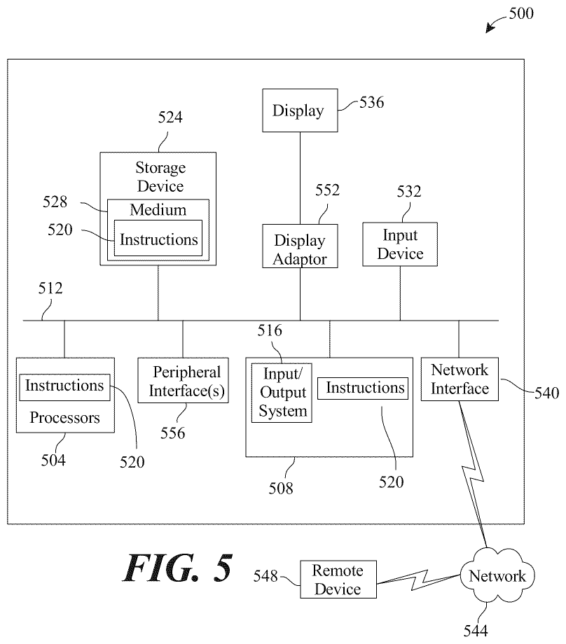

Further referring to FIG. 3, manufacturing device 300 may include an automated manufacturing system. In some embodiments, an automated manufacturing system is a manufacturing device 300 including a controller 312 that controls one or more manufacturing steps automatically. Controller 312 may include a sequential control device that produces a sequence of commands without feedback from other components of automated manufacturing system. Controller 312 may include a feedback control device that produces commands triggered or modified by feedback from other components. Controller 312 may perform both sequential and feedback control. In some embodiments, controller 312 includes a mechanical device. In other embodiments, controller 312 includes an electronic device. Electronic device may include digital or analog electronic components, including without limitation one or more logic circuits, such one or more logic gates, programmable elements such as field-programmable arrays, multiplexors, one or more operational amplifiers, one or more diodes, one or more transistors, one or more comparators, and one or more integrators. Electronic device may include a processor. Electronic device may include a computing device 500 as described below in reference to FIG. 5. Computing device 500 may include a computing device 500 embedded in manufacturing device 300; as a non-limiting example, computing device 500 may include a microcontroller 312, which may be housed in a unit that combines the other components of manufacturing device 300. Controller 312 may include a manufacturer client of plurality of manufacturer clients; controller 312 may be communicatively coupled to a manufacturer client of plurality of manufacturer clients.

Continue referring to FIG. 3, controller 312 may include a component embedded in manufacturing device 300; as a non-limiting example, controller 312 may include a microcontroller 312, which may be housed in a unit that combines the other components of manufacturing device 300. Further continuing the example, microcontroller 312 may have program memory, which may enable microcontroller 312 to load a program that directs manufacturing device 300 to perform an automated manufacturing process. Similarly, controller 312 may include any other components of a computing device 500 as described below in reference to FIG. 3 in a device housed within manufacturing device 300. In other embodiments, controller 312 includes a computing device 500 that is separate from the rest of the components of manufacturing device 300; for instance, controller 312 may include a personal computer, laptop, or workstation connected to the remainder of manufacturing device 300 by a wired or wireless data connection. In some embodiments, controller 312 includes both a personal computing device 500 where a user may enter instructions to generate a program for turning workpiece into a finished product, and an embedded device that receives the program from the personal computing device 500 and executes the program. Persons skilled in the art will be aware of various ways that a controller 312, which may include one or more computing devices, may be connected to or incorporated in an automated manufacturing system as described above.

Still referring to FIG. 3, controller 312 may control components of automated manufacturing system; for instance, controller 312 may control elements including without limitation tool changer to switch endmills, spindle or gear systems operatively coupled to spindle to regulate spindle rotational speed, linear movement of applicator 304, base table, or both, and rotation or rotational position of rotary table. As an example, applicator 304 may be moved about using computerized numerical control (CNC) devices and/or motion controls that are automated and operate by precisely programmed commands that control movement of one or more parts of the equipment to affect the material removal. CNC machines, their operation, programming, and relation to computer aided manufacturing (CAM) tools and computer aided design (CAD) tools are well known and need not be described in detail herein for those skilled in the art to understand the scope of the present invention and how to practice it in any of its widely varying forms. Similarly, controller 312 may coordinate deposition and/or curing of material in additive manufacturing processes, where manufacturing device 300 is an additive manufacturing device 300. Persons skilled in the art, upon reading the entirety of this disclosure, will be aware of similar automated control systems usable for various forms manufacturing. Controller may be, be included in, include, and/or be in communication with computing device 500.

Further referencing on FIG. 3, in operation, the manufacturing device 300 may deposit layers of encrypted nucleic acid sample 152, as programmed by computing device 500 and/or controller 312; such programming may be driven, in turn, by codebook 120 generated as a function of secret 116 defined by subject 108 using computing device 500 and/or controller 312. In an embodiment, manufacturing device 300 may print encrypted nucleic acid sample 152 onto a chip, wherein the chip may be any chip described in this disclosure as reference to illumina dye sequencing technique in FIG. 1.

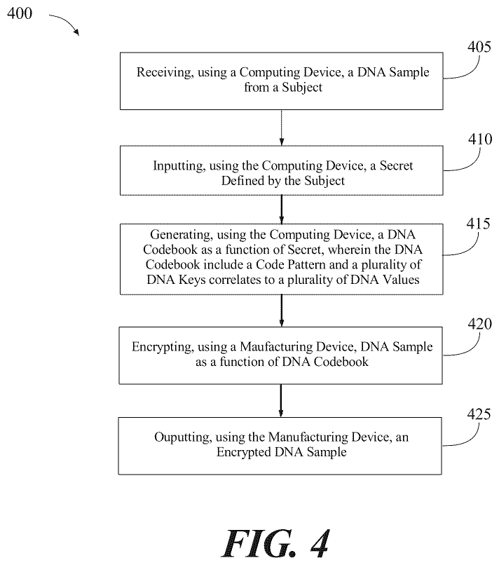

Now referring to FIG. 4, an exemplary method 400 of physically encrypting nucleic acid is illustrated. Method 400 includes a step 405 of receiving a nucleic acid sample from a subject, without limitation, as described above in reference to FIGS. 1-3. In some embodiments, nucleic acid sample may include sample taken from drinking glasses, food with bite marks, bite marks on skin, oral buccal swabbing, and the like thereof. This may be implemented, without limitation, as described above in reference to FIGS. 1-3.

With continued reference to FIG. 4, method 400 includes a step 410 of inputting, using the computing device, a secret defined by the subject, without limitation, as described above in reference to FIGS. 1-3. In some embodiments, secret may be kept unknown and/or unseen other than subject. In some embodiments, secret may be a string defined by subject. In other embodiments, secret may include a sequence length limitation. This may be implemented, without limitation, as described above in reference to FIGS. 1-3.

With continued reference to FIG. 4, method 400 includes a step 415 of generating, using the computing device, a nucleic acid codebook as a function of secret wherein the nucleic acid codebook includes a code pattern, and a plurality of nucleic acid keys correlates to a plurality of nucleic acid values. This may be implemented, without limitation, as described above in reference to FIGS. 1-3. In some cases, nucleic acid code book may include a one-time codebook. In some embodiments, code pattern may include a codeword, a plaintext, and a STOP codon configured to terminate the encryption. This may be implemented, without limitation, as described above in reference to FIGS. 1-3. In some embodiments, each nucleic acid key of plurality of nucleic acid keys may include a nucleic acid sequence element of nucleic acid sample. In some embodiments, each nucleic acid value of plurality of nucleic acid values may include an encrypted code. This may be implemented, without limitation, as described above in reference to FIGS. 1-3.

With continued reference to FIG. 4, method 400 includes a step 420 of encrypting, using a manufacturing device, nucleic acid sample as a function of nucleic acid codebook, without limitation, as described above in reference to FIGS. 1-3. In some embodiments, encrypting nucleic acid sample may include encrypting nucleic acid sample as a function of CRISPR. In other embodiments, encrypting nucleic acid sample may include encrypting nucleic acid sample as a function of illumina dye sequencing technique. This may be implemented, without limitation, as described above in reference to FIGS. 1-3.

With continued reference to FIG. 4, method 400 includes a step 425 of outputting, using the manufacturing device, an encrypted nucleic acid sample, without limitation, as described above in reference to FIGS. 1-3. In some embodiments, outputting the encrypted nucleic acid sample may include decrypting the encrypted nucleic acid sample as a function of a nucleic acid decryption method. In some embodiments, nucleic acid decryption method may include purifying the encrypted nucleic acid sample using a plurality of magnetic beads. In some embodiments, nucleic acid decryption method may further include annealing plurality of magnetic beads. In some cases, annealing plurality of magnetic beads may include heating plurality of magnetic beads at a first temperature and cooling plurality of magnetic beads at a second temperature, wherein the second temperature is different than the first temperature. In some embodiments, nucleic acid decryption method may further include producing an encrypted nucleic acid sequence as a function of the plurality of magnetic beads. In some embodiments, nucleic acid decryption method may further include mapping encrypted nucleic acid sequence to a decrypted nucleic acid sequence as a function of nucleic acid codebook. This may be implemented, without limitation, as reference to FIGS. 1-3.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

FIG. 5 shows a diagrammatic representation of one embodiment of a computing device in the exemplary form of a computer system 500 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 500 includes a processor 504 and a memory 508 that communicate with each other, and with other components, via a bus 512. Bus 512 may include any of several types of bus structures including, but not limited to, a memory bus, a memory controller, a peripheral bus, a local bus, and any combinations thereof, using any of a variety of bus architectures.

Processor 504 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 504 may be organized according to Von Neumann and/or Harvard architecture as a non-limiting example. Processor 504 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 508 may include various components (e.g., machine-readable media) including, but not limited to, a random-access memory component, a read only component, and any combinations thereof. In one example, a basic input/output system 516 (BIOS), including basic routines that help to transfer information between elements within computer system 500, such as during start-up, may be stored in memory 508. Memory 508 may also include (e.g., stored on one or more machine-readable media) instructions (e.g., software) 520 embodying any one or more of the aspects and/or methodologies of the present disclosure. In another example, memory 508 may further include any number of program modules including, but not limited to, an operating system, one or more application programs, other program modules, program data, and any combinations thereof.

Computer system 500 may also include a storage device 524. Examples of a storage device (e.g., storage device 524) include, but are not limited to, a hard disk drive, a magnetic disk drive, an optical disc drive in combination with an optical medium, a solid-state memory device, and any combinations thereof. Storage device 524 may be connected to bus 512 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 524 (or one or more components thereof) may be removably interfaced with computer system 500 (e.g., via an external port connector (not shown). Particularly, storage device 524 and an associated machine-readable medium 528 may provide nonvolatile and/or volatile storage of machine-readable instructions, data structures, program modules, and/or other data for computer system 500. In one example, software 520 may reside, completely or partially, within machine-readable medium 528. In another example, software 520 may reside, completely or partially, within processor 504.

Computer system 500 may also include an input device 532. In one example, a user of computer system 500 may enter commands and/or other information into computer system 500 via input device 532. Examples of an input device 532 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 532 may be interfaced to bus 512 via any of a variety of interfaces (not shown) including, but not limited to, a serial interface, a parallel interface, a game port, a USB interface, a FIREWIRE interface, a direct interface to bus 512, and any combinations thereof. Input device 532 may include a touch screen interface that may be a part of or separate from display 536, discussed further below. Input device 532 may be utilized as a user selection device for selecting one or more graphical representations in a graphical interface as described above.

A user may also input commands and/or other information to computer system 500 via storage device 524 (e.g., a removable disk drive, a flash drive, etc.) and/or network interface device 540. A network interface device, such as network interface device 540, may be utilized for connecting computer system 500 to one or more of a variety of networks, such as network 544, and one or more remote devices 548 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 544, may employ a wired and/or a wireless mode of communication. In general, any network topology may be used. Information (e.g., data, software 520, etc.) may be communicated to and/or from computer system 500 via network interface device 540.

Computer system 500 may further include a video display adapter 552 for communicating a displayable image to a display device, such as display device 536. Examples of a display device include, but are not limited to, a liquid crystal display (LCD), a cathode ray tube (CRT), a plasma display, a light emitting diode (LED) display, and any combinations thereof. Display adapter 552 and display device 536 may be utilized in combination with processor 504 to provide graphical representations of aspects of the present disclosure. In addition to a display device, computer system 500 may include one or more other peripheral output devices including, but not limited to, an audio speaker, a printer, and any combinations thereof. Such peripheral output devices may be connected to bus 512 via a peripheral interface 556. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method of nucleic acid physical encryption, wherein the method comprises:
receiving a nucleic acid sample from a subject;
inputting, using a computing device, a secret defined by the subject;
generating, using the computing device, a nucleic acid codebook as a function of the secret, wherein the nucleic acid codebook comprises:
a code pattern generated using the secret; and
a plurality of nucleic acid keys correlating to a plurality of nucleic acid values;
encrypting, using a manufacturing device, the nucleic acid sample as a function of the nucleic acid codebook, wherein encrypting further comprises replacing at least a sequence of the sample with a code pattern by matching a key; and
outputting, using the manufacturing device, an encrypted nucleic acid sample.

2. The method of claim 1, wherein the code pattern comprises:
a codeword;
a plaintext; and
a STOP codon configured to terminate the encryption.

3. The method of claim 1, wherein each nucleic acid key of the plurality of nucleic acid keys comprises a nucleic acid sequence element of the nucleic acid sample.

4. The method of claim 1, wherein each nucleic acid value of the plurality of nucleic acid values comprises an encrypted code.

5. The method of claim 1, wherein nucleic acid codebook comprises a one-time codebook.

6. The method of claim 1, wherein encrypting the nucleic acid sample comprises encrypting the nucleic acid sample as a function of CRISPR.

7. The method of claim 1, wherein encrypting the nucleic acid sample comprises encrypting the nucleic acid sample as a function of illumina dye sequencing technique.

8. The method of claim 1, wherein outputting the encrypted nucleic acid sample comprises decrypting the encrypted nucleic acid sample as a function of a nucleic acid decryption method.

9. The method of claim 8, wherein the nucleic acid decryption method comprises:
purifying the encrypted nucleic acid sample using a plurality of magnetic beads;
annealing the plurality of magnetic beads;
producing an encrypted nucleic acid sequence as a function of the plurality of magnetic beads; and
mapping the encrypted nucleic acid sequence to a decrypted nucleic acid sequence as a function of the nucleic acid codebook.

10. The method of claim 9, wherein annealing the plurality of magnetic beads comprises:
heating the plurality of magnetic beads at a first temperature; and
cooling the plurality of magnetic beads at a second temperature;
wherein the second temperature is different than the first temperature.

11. An apparatus of nucleic acid physical encryption, wherein the system comprises:
at least a computing device configured to:
receive a nucleic acid sample from a subject;
input a secret defined by the subject;
generate a nucleic acid codebook as a function of the secret, wherein the nucleic acid codebook comprises:
a code pattern; and
a plurality of nucleic acid keys correlates to a plurality of nucleic acid values; and
at least a manufacturing device configured to:
encrypt a nucleic acid sample received from a subject as a function of the nucleic acid codebook; and
output an encrypted nucleic acid sample.

12. The method of claim 1, wherein the code pattern comprises:
a codeword;
a plaintext; and
a STOP codon configured to terminate the encryption.

13. The apparatus of claim 11, wherein each nucleic acid key of the plurality of nucleic acid keys comprises nucleic acid sequence element of the nucleic acid sample.

14. The apparatus of claim 11, wherein each nucleic acid value of the plurality of nucleic acid values comprises an encrypted code.

15. The apparatus of claim 11, wherein nucleic acid codebook comprises a one-time codebook.

16. The apparatus of claim 11, wherein encrypting the nucleic acid sample comprises encrypting the nucleic acid sample as a function of CRISPR.

17. The apparatus of claim 11, wherein encrypting the nucleic acid sample comprises encrypting the nucleic acid sample as a function of illumina dye sequencing technique.

18. The apparatus of claim 11, wherein outputting the encrypted nucleic acid sample comprises decrypting the encrypted nucleic acid sample as a function of a nucleic acid decryption method.

19. The apparatus of claim 18, wherein the nucleic acid decryption method comprises:
purifying the encrypted nucleic acid sample using a plurality of magnetic beads;
annealing the plurality of magnetic beads;
producing an encrypted nucleic acid sequence as a function of the plurality of magnetic beads; and
mapping the encrypted nucleic acid sequence to a decrypted nucleic acid sequence as a function of the nucleic acid codebook and the secret.

20. The apparatus of claim 19, wherein annealing the plurality of magnetic beads comprises:
heating the plurality of magnetic beads at a first temperature; and
cooling the plurality of magnetic beads at a second temperature;
wherein the second temperature is different than the first temperature.
